(19) United States
(12) Patent Application Publication
(10) Pub. No.: US 2012/0070536 A1
(43) Pub. Date: Mar. 22, 2012

(54) LACTOBACILLUS STRAIN AND FOOD HAVING ANTIFUNGAL ACTIVITY

(75) Inventors: Naomi Kokubo, Tokyo (JP); Miyuki Ozawa, Tokyo (JP); Seigo Nakaya, Tokyo (JP); Azusa Kato, Tokyo (JP); Shinichiro Ichinose, Tokyo (JP); Shiro Sasaki, Tokyo (JP)

(73) Assignees: WAKAMOTO PHARMACEUTICAL CO., LTD., TOKYO (JP); MITSUI & CO., LTD., TOKYO (JP)

(21) Appl. No.: 13/258,737

(22) PCT Filed: Apr. 13, 2010
(86) PCT No.: PCT/JP2010/056618

§ 371 Date: Dec. 6, 2011

(30) Foreign Application Priority Data
Apr. 15, 2009 (JP) 2009-099033

(51) Int. Cl. C12N 1/20; A21D 13/00; A21D 8/04; C12P 1/04

(52) U.S. Cl. 426/20; 435/252.9; 426/61; 426/549

(57) ABSTRACT

An object of the present invention is to provide a novel strain that is capable of effectively inhibiting the growth of microorganisms such as fungi and Staphylococcus aureus, is safe, and does not influence the flavor and taste of foods. The present invention relates to a strain of Lactobacillus sanfranciscensis WB1006 (FERM ABP-11246), and also relates to a food produced with the use of the strain.

### TECHNICAL FIELD

The present invention relates to a novel strain of Lactobacillus sanfranciscensis; a culture of the strain, a material containing the strain or a lyophilized powder of the strain and a food, such as bread, produced with the use of the strain.

### BACKGROUND ART

Bread shows quality deterioration and loses its commercial value with the growth of fungi. Since bread products are transported and sold at ambient temperature, antifungal agents are used in some bread products although bread is generally made in a clean environment. Examples of antifungal agents include those mainly composed of an acidulant such as acetic acid and citric acid, or alternatively a pH adjuster such as sodium acetate, and of a bacteriostatic agent such as propionic acid and ethanol. These additives problematically add acidity, acidic or alcoholic smell, and the like to the original flavor and taste of bread. This problem as well as the recent increasing interest in natural food have created a trend away from these chemical antifungal agents, and led to the production of bread with an antifungal effect using lactic acid bacteria, which are considered to be safer than other microorganisms based on the long history of their usage (Patent Document 1).

Dough starters for bread with sour taste, which are obtained by fermentation of such lactic acid bacteria and yeasts, are called sourdough'starters. San Francisco sour bread in the west coast of U.S. and panettone in Italy are famous as breads made with use of these starters. For example, Lactobacillus sanfranciscensis, Lactobacillus plantarum, Lactobacillus brevis, Lactobacillus casei, and Lactobacillus fermentum are commonly known as lactic acid bacteria isolated from the sourdough starters of these breads (Non-Patent Document 1). Novel strains have also been reported: Lactobacillus comoensis (Patent Document 2) and Lactobacillus acidifarinarius (Patent Document 3).

Panettone starters are a very peculiar type of sourdough starters. Cultivation of these bread starters is thought to be possible only in specific regions in Italy. For example, Lactobacillus comoensis lives in panettone starters in symbiosis with other lactic acid bacteria and yeasts, and still today, the panettone starters are maintained by daily subculture in according with a traditional method in northern Italy (Patent Document 4).

Breads made with use of panettone starters are known to have effects such as antifungal and preservative effects without any preservatives. It is known that these effects are given by lactic acid bacteria present in the panettone starters and fermentation products of these bacteria and the like (Patent Document 5).

For foods and beverages such as bread, the use of a lipase treatment product of a reaction product obtained by reacting cells of a lactic acid bacterium and a fat in an aqueous medium is known as a method for producing an antifungal effect (Patent Document 6). For alcoholic beverages and the like, a culture of Streptomyces fulvissimus FERN P-16347 is used as an antibacterial and anticaries agent for food. This bacterium is also known to be effective in inhibiting the growth of Staphylococcus aureus, which is a food poisoning bacterium (Patent Document 7).

Lactobacillus acidophilus L-55 is commonly known to have an ability to produce an antibacterial substance against bacteria including Escherichia coli, Staphylococcus aureus, Bacillus subtilis, and Listeria monocytogenes, and is generally recognized as a lactic acid bacterium usable for yogurt and other foods (Patent Document 8). However, its effect on bread is still unknown.

Traditional panettone starters are a culture of microorganisms, such as lactic acid bacteria and yeasts, which spontaneously adhere to and grow on a medium containing flour and the like in a peculiar climate and environment specific to each region. These starters are typically maintained by subculture. Accordingly, the bacterial flora of panettone starters is peculiar to the climate and environment of the regions. In conventional studies, a specific lactic acid bacterium was isolated from such a panettone starter and cultured, and then inoculated in a liquid or dough medium mainly composed of flour to cause fermentation (Patent Documents 9 and 10). Commercial products thereof are available.

In sourdough starters, several lactic acid bacteria and yeasts live together in symbiosis. This microbial environment is difficult to artificially replicate and allows these microorganisms to exert their performance. It is thought that these starters can be stably subcultured only in their specific regions or environments, and are very susceptible to changes in factors such as region and handling manner. Sometimes, these starters also lose their function as starters, as a result of contamination and proliferation of, for example, a foreign microorganism that is not originally present in the starters.

In the case where a pure culture of a lactic acid bacterium isolated from a sourdough starter is used as a starter, the stability of the quality of a first-generation sourdough starter is enhanced but, with repeated subculture, the characteristics of the successive sourdough starters are likely to change from those of the first-generation starter because of the same reason (Patent Document 11).

In addition, in the case of a pure culture of a lactic acid bacterium for sourdough starters, some bacteria require laborious operations such as the preparation of a special nutrient medium. For example, several nutrient media for Lactobacillus sanfranciscensis, which is one of representative lactic acid bacteria for sourdough starters, are known and examples of their disadvantages are as follows: (1) preparation of these media is laborious; (2) variations in the culture yield caused by the lot-lot difference in the medium components are large; and (3) these media do not allow sufficient growth of the bacterium and therefore are not suited for large-scale culture. Commercial products of dough starters for bread are in a liquid or dough, form. One disadvantage of these products is that their performance varies depending on the storage conditions even within their shelf life.

Staphylococcus aureus, which is known as a food-poisoning bacterium, is found on human skin, wound on the skin, and the like. Accordingly, this bacterium often causes food poisoning through foods, such as cooked bread, which are prepared by hand. Staphylococcus aureus produces a heat-resistant toxin in the course of growth, which is thought to cause food poisoning. Since the activity of the toxin is not lost even if bacterial cells are killed by heating, it is important to inhibit the growth of the bacterium itself in order to prevent food poisoning. However, it has not been known how to effectively inhibit the growth of Staphylococcus aureus.

Patent Document 1: JP-A 2004-081212

Patent Document 2: JP-A S63-146742

Patent Document 3: JP-A 2003-169680

Patent Document 4: JP-A S63-112977

Patent Document 5: JP-A 2002-291466

Patent Document 6: WO 2005/104879

Patent Document 7: JP-A 2002-000261

Patent Document 8: JP-A 2003-230376

Patent Document 9: JP-A 2006-158382

Patent Document 10: JP-A 2007-244274

Patent Document 11: JP-A H5-252937

Non-Patent Document 1: “Science and Technology of Lactic Acid Bacteria”, Nyuusankin Kenkyushuudankai Edition, Japan Scientific Societies Press, April, 1996

### SUMMARY OF THE INVENTION

### Problems to be Solved by the Invention

Bread making materials using commercial lactic acid bacteria cannot always produce a sufficient antifungal effect. Therefore, there is a strong need for search of a novel lactic acid bacterium that is capable of effectively inhibiting the growth of microorganisms such as fungi, is safe and has less influence on the flavor and taste of foods, and development of products of the same. Since it has been reported that the antifungal effect of some lactic acid bacteria varies depending on symbiotic yeast, there is also a need for a bacterium whose antifungal effect does not vary even if a widely-used commercial yeast is used together. Accordingly, it is important to develop a product, such as a long-life lyophilized powder, of a lactic acid bacterium that is expected to produce a sufficient antifungal effect, and to construct a bread making method using fermentation by this bacterium.

### Means for Solving the Problems

The present invention aims to solve the above problems and relates to a novel strain of Lactobacillus sanfranciscensis WB1006 (FERN ABP-11246), which has been found by the present inventors.

The present invention also relates to a strain having the following bacteriological characteristics (1) to (9):

(1) gram positive;

(2) rod shaped;

(3) nonmotile;

(4) nonsporing;

(5) facultative anaerobe;

(6) catalase negative;

(7) a growth temperature range of from 10° C. to 28° C.;

(8) a growth pH range of from 5.5 to 9.0; and

(9) utilizing maltose to produce D(+)- and L(+)-lactic acids, ethanol, and carbon dioxide.

The present invention also relates to a culture of the strain, a material containing the strain, or a lyophilized powder of the strain. Preferably, the strain is viable.

The present invention also relates to a method for culturing a strain, which includes: inoculating the above-mentioned strain in a medium; and culturing the strain.

The present invention also relates to a method for producing a food, which includes fermentation by the culture, the material or the lyophilized powder, and a food produced by this production method.

The present invention also relates to a food additive for inhibiting the growth of fungi and Staphylococcus aureus, which contains the strain, the culture, the material, or the lyophilized powder, as an active ingredient.

Preferably, the food additive is still capable of inhibiting the growth of fungi and Staphylococcus aureus after heating treatment.

The present invention also relates to a food containing the food additive.

Preferably, the food is obtained by fermenting a food material by the strain.

The present invention also relates to a method for producing a starter for the purpose of producing a growth inhibitory effect against fungi and Staphylococcus aureus, which includes fermenting a mixture containing a lactic acid bacterium, flour and water, wherein the lactic acid bacterium is a lactic acid bacterium that mainly utilizes one sugar other than glucose and slightly utilizes another sugar.

Preferably, in the production method, the mixture further contains a yeast.

Preferably, in the production method, the lactic acid bacterium is capable of growing at a temperature of 10° C. to 15° C.

The present invention also relates to a method for producing a food, which includes: producing a sponge using a starter produced by the above-mentioned method; and forming a final dough.

### EFFECTS OF THE INVENTION

Foods, such as bread, produced using Lactobacillus sanfranciscensis WB1006 of the present invention have an enhanced antifungal effect, an enhanced growth inhibitory effect against Staphylococcus aureus, and a good flavor, taste and texture, as compared to bread produced using Lactobacillus sanfranciscensis or Lactobacillus plantarum, which are known to have an antifungal effect. Especially, in the case where the strain of the present invention is used in bread, its growth inhibitory effect against fungi and Staphylococcus aureus is maintained even after heating treatment such as a process of baking a bread dough, and a process of preparing a cooked bread. Accordingly, when used in a bread dough, the strain of the present invention can prevent the growth of Staphylococcus aureus itself even in a bread made through processes at high temperatures such as baking and cooking, and therefore can fundamentally prevent production of the heat-resistant toxin, which is difficult to decompose and remove.

In addition, when the strain of the present invention is used in the form of a lyophilized powder that eliminates the necessity of subculture and is easy to store, it enables production of a stable starter. A great effect of the strain Lactobacillus sanfranciscensis WB1006 of the present invention is to facilitate achieving the long-life characteristic and antifungal effect of panettone which has been produced by the traditional method in northern Italy.

### BRIEF DESCRIPTION OF THE DRAWINGS

FIG. 1 is a graph showing a temporal change in the number of spots where sporulation of Aspergillus niger was observed in the breads made in Comparative Examples 1 and 2, and Example 1;

FIG. 2 is a graph showing a temporal change in the number of spots where sporulation of Penicillium chrysogenum was observed in the breads made in Comparative Example 3 and Example 2; and

FIG. 3 is a graph of the growth ratio of Staphylococcus aureus in the breads made in Comparative Example 3 and Example 2.

### MODES FOR CARRYING OUT THE INVENTION

### (I) Strain

The strain Lactobacillus sanfranciscensis WB1006 of the present invention was deposited on Oct. 29, 2008 under accession number FERM P-21711 in International Patent Organism Depositary, independent administrative institution, National Institute of Advanced Industrial Science and Technology, and was internationally deposited on Apr. 8, 2010 under accession number FERM ABP-11246.

Lactobacillus sanfranciscensis WB1006 of the present invention is a lactic acid bacterium isolated and discovered from a panettone starter for bread making. The strain WB1006 produces an excellent antifungal effect as compared to other several lactic acid bacteria present in panettone starters. It has been thought that the flavor, taste and texture peculiar to panettone and its long-life characteristic are given by a combination of several lactic acid bacteria; however, even the strain WB1006 alone can provide these characteristics.

The following shows the bacteriological characteristics of Lactobacillus sanfranciscensis WB1006 of the present invention.

(1) Gram positive

(2) Rod shaped

(3) Nonmotile

(4) Nonsporing

(5) Facultative anaerobe

(6) Catalase negative

(7) Growth temperature: 10° C. to 28° C. (optimum growth temperature 25° C.)

(8) Growth pH: 5.5 to 9.0

(9) The strain utilizes maltose to produce D(+)- and L(+)-lactic acids, ethanol, and carbon dioxide.

Also, the strain very slightly utilizes glucose under some culture conditions.

The strain of the present invention is considered to belong to obligately heterofermentative lactobacilli because it is a gram positive, nonsporing, facultatively anaerobic, and catalase negative bacillus and produces lactic acid and ethanol from a sugar.

The following shows the cultural characteristics of the strain of the present invention.

(1) 1% Maltose-containing MRS agar plate medium

Colonies, after 3- or 4-day culture at 25° C., are circular, approximately 2 to 3 mm or less in diameter, convex in elevation, opaque grayish-white in color, and slightly dry.

(2) MYP liquid medium

After 24-hour culture at 25° C., cells grow to make the medium cloudy and to form a fluffy precipitate in the medium.

(3) MYP agar medium (stab culture)

Cells uniformly grow in a puncture in the medium.

The following shows the physiological and biochemical characteristics of the strain of the present invention.

(1) Growth temperature: 10° C. to 28° C. (optimum growth temperature 25° C.)

(2) Growth pH range: 5.5 to 9.0

(3) Relationship with oxygen:

Facultatively anaerobic. The strain can grow either in the presence of oxygen or under anaerobic conditions.

(4) Substances essential for growth:

Maltose, yeast extract and a fatty acid, in particular an unsaturated fatty acid are essentially required in the MYP liquid medium.

(5) Sugar fermentability:

The strain utilizes maltose to produce an acid and gas.

(6) Litmus milk: no change

(7) Reduction of nitrates: negative

(8) Gelatin is not liquefied.

(9) Urease: negative

(10) Catalase: negative

(11) Starch is not hydrolyzed.

(12) Products from maltose: L(+)- and D(+)-lactic acids and ethanol

A comparison of the sugar utilization between the strain of the present invention and Lactobacillus sanfranciscensis JCM5668 shows that the strain of the present invention remarkably specifically utilizes maltose, and hardly utilizes glucose under common culture conditions; and Lactobacillus sanfranciscensis JCM5668, on the other hand, utilizes both maltose and glucose.

Based on 16S rRNA gene analysis, the 1564 by sequence of the strain of the present invention preferably has 97% or higher, and more preferably 99% or higher sequence identity to Lactobacillus sanfranciscensis JCM5668 (JAPAN COLLECTION OF MICROORGANISMS, independent administrative institution, RIKEN), which is the type strain of Lactobacillus sanfranciscensis.

In the present invention, the strain is used in the form of a culture of the strain, a material containing the strain or a lyophilized powder of the strain. The strain of the present invention is available from a panettone starter and, for example, can be isolated from a panettone starter for bread making by a known technique. For example, a panettone starter is gradually diluted with sterilized saline, and applied (inoculated) to and incubated in an isolation agar medium (e.g. MYP agar medium, 1% maltose-containing MRS agar medium) containing 10 ppm of cycloheximide and 10 ppm of sodium azide. Then, the strain can be isolated by separating colonies in the medium.

The strain of the present invention can be cultured without any procedure after inoculation of the strain in a medium. The strain of the present invention enables the characteristics of a first-generation sourdough to be successively maintained without daily subculture, and therefore allows easy cultivation. The strain of the present invention does not require a special nutrient medium, which leads to easy preparation. Thus, variations in the culture yield, which are caused by the lot-lot difference in the medium components, can be avoided, and the strain of the present invention can be mass cultured. Here, the culture medium used may be an agar medium or a liquid medium based on the purpose. The medium preferably contains yeast extract and Tween 80 to accelerate the growth of the strain of the present invention. The culture temperature is 10° C. to 28° C., preferably 23° C. to 28° C. and more preferably 25° C. The growth pH is 5.5 to 9.0, and a pH of 5.5 to 7.0 is preferable. The culture period is preferably 2 to 4 days. The culture temperature range of the strain of the present invention is 28° C. or lower, and thus is lower than the culture temperature range of Lactobacillus sanfranciscensis JCM5668, 30° C. to 35° C. This feature is beneficial because no special heat-insulation facility is required. Although the strain of the present invention may be cultured alone, it can be cultured with several kinds of yeasts since the strain does not influence the growth of other microorganisms. Examples of yeasts include Saccharomyces cerevisiae and Saccharomyces exiguus.

The culture of the strain refers to a culture obtained by culturing the strain itself.

The material containing the strain refers to a powdery, liquid, dough or solid product containing the strain.

The lyophilized powder of the strain refers to a powder that is obtained by rapidly freezing the strain and sublimating moisture at a reduced pressure. The temperature for freezing the strain is preferably −50° C. to −80° C., and the pressure is preferably reduced to 15 to 100 Pa.

The strain is preferably viable in the culture of the strain, the material containing the strain or the lyophilized powder of the strain because a substance (fermentation product) produced in the growth process of the lactic acid bacterium is usable.

### (II) Usage of Strain (Food Additive)

A food additive can be produced by using the strain of the present invention, the culture of the strain, the material containing the strain or the lyophilized powder of the strain as an active ingredient. The food additive of the present invention means an additive used for a specific purpose in the process of food production or storage and is not particularly limited, provided that it is added in foods, such as bread, as described below. The food additive of the present invention may be, for example, an antibacterial agent, antifungal agent or the like.

The food additive of the present invention produces an effect of inhibiting the growth of fungi and Staphylococcus aureus in food. The term “inhibiting the growth” means to inhibit cell division.

The food additive of the present invention produces a growth inhibitory effect against a wide variety of fungi, and inhibits the growth of fungi of the genera: Aspergillus, Penicillium, Arthrinium, Acremonium, Alternaria, Exophiala, Epicoccum, Aureobasidium, Curvularia, Cladosporium, Chaetomium, Geotrichum, Sporothrix, Trichoderma, Trichophyton, Drechslera, Nigrospora, Neurospora, Pichia, Pithomyces, Phialophora, Phoma, Fusarium, Paecillomyces, Pestalotiopsis, Botrytis, Mucor, Monascus, Monilliera, Eurotium, Rhodotorula, and Wallemia. The growth inhibitory effect is more effective against species of Aspergillus and Penicillium among these, and remarkably effective against Aspergillus niger and Penicillium chrysogenum. The food additive of the present invention also produces a strikingly strong growth inhibitory effect against Staphylococcus aureus and Candida albicans.

The form of the food additive of the present invention is not particularly limited and may be any of powder, liquid and solid. The food additive of the present invention contains the strain of the present invention, the culture of the strain, the material containing the strain, or the lyophilized powder of the strain, as an active ingredient, and may further contain other ingredients. Examples of other ingredients include those commonly used in food processing agents, preservatives, antioxidants, antibacterial agents, antifungal agents, and the like. In the case where the food additive is in a solid form, it may contain, but not limited to, a filler, a binder, a processing aid or the like.

The growth inhibitory effect of the food additive of the present invention against fungi and Staphylococcus aureus will not disappear even after heating treatment on foods. The heating treatment is not particularly limited and may be any of baking in an oven, heating at an elevated pressure, and wet heat treatment. The heating treatment herein means a heating treatment in which a heating target is heated to 80° C. or higher, and preferably to 90° C. or higher.

### (III) Food Production Method

The strain of the present invention can be used to produce a food. The food is not particularly limited and preferred examples thereof include various bakery products such as bread, Danish pastry, and panettone; fresh confections such as cake and waffle; semi-fresh confections such as madeleine and financier; and dry confections such as cookies, because the production thereof involves fermentation that efficiently produces fermentation products.

The method for making a bread or confection is not particularly limited and is preferably a method which accelerates fermentation by the strain of the present invention in the making process and allows production and accumulation of the fermentation products in the food. Any bread making methods can be performed using the strain or the material containing the strain, but for example, a sponge and dough method using the material containing the strain is preferable for inhibition of the growth of fungi.

Specifically, the sponge and dough method includes: adding a starter to a sponge and allowing the resulting sponge to ferment; adding the rest of ingredients and forming a final dough through kneading, shaping and fermenting; and baking and cooling the final dough.

In the present invention, the term “starter” refers a mixture containing flour, water, and a lactic acid bacterium that has been kneaded and allowed to ferment. Although the mixture may further contain a yeast, the starter can produce a sufficient effect without yeasts. In the present invention, the yeast is not particularly limited and Saccharomyces cerevisiae, which is commonly used in bread making, can be used.

The method for producing a starter is not particularly limited and is preferably a method that accelerates successful production of fermentation products by the lactic acid bacterium to inhibit the growth of fungi and Staphylococcus aureus. Based on common bread making methods, the proportion of water to flour in the starter is preferably from 50 to 120, taking the amount of the flour as 100. The proportion of the bacterial powder of the present invention to flour in the starter is preferably from 1 to 2, taking the amount of the flour as 100, because the strain of the present invention can successfully produce fermentation products. The proportion of yeast to flour in the starter is preferably from 0.1 to 0.2, taking the amount of the flour as 100, because the yeast adequately causes fermentation, which results in a bread with good flavor and taste.

The production process of the starter is not particularly limited. The temperature of kneading the mixture is preferably 18° C. to 32° C., and more preferably 20° C. to 30° C., considering the activity of the lactic acid bacterium. The. fermentation temperature after kneading the mixture is preferably 18° C. to 32° C., and more preferably 25° C. to 30° C. to achieve the maximum activity of the lactic acid bacterium. The humidity for fermenting the mixture is preferably 50 to 100 RH%, and more preferably 70 to 80 RH % to prevent the dough surface from becoming dry. The time period for fermenting the mixture is preferably 8 to 48 hours, and more preferably 12 to 24 hours for sufficient growth of the bacterium.

The lactic acid bacterium that causes fermentation of the starter is preferably a lactic acid bacterium that has a bacteriological characteristic of mainly utilizing one sugar other than glucose and slightly utilizing sugars including glucose but other than the above sugar because such a lactic acid bacterium does not compete for the nutrients against yeasts and produces a sufficient effect in the production process of a fermented food. In order to obtain a fermentation product having a storage stability effect, the lactic acid bacterium preferably has a bacteriological characteristic of being capable of growing at a temperature of 10° C. to 15° C. The lactic acid bacterium is more preferably a lactic acid bacterium that mainly utilizes maltose and slightly utilizes glucose, and Lactobacillus sanfranciscensis WB1006 is particularly preferable. The characteristic of mainly utilizing maltose and slightly utilizing glucose means that maltose is required as a main carbon source and the growth is hardly susceptible to lack of glucose. Specifically, it means that the growth is readily observed after 24-hour culture in a maltose-containing medium, and the growth is only slightly confirmed by visual observation of cells collected by centrifugation after 48-hour or longer culture in a glucose-containing medium.

The method for making a sponge and the method of adding the starter to the sponge and allowing the resulting sponge to ferment are not particularly limited, and may be a sponge and dough method commonly used in bread making. The rest of ingredients that are added after fermentation of the sponge containing the starter are not particularly limited and examples thereof include materials commonly used in bread making such as salt, sugar, skim milk powder, shortening, water, bread improvers, and dairy products. The kneading, dividing, shaping, fermenting, baking and cooling processes are not limited to specific processes and can be performed by a sponge and dough method commonly used in bread making.

In the case where the strain of the present invention is prepared in the form of a lyophilized powder and used for a bread starter, the above-mentioned MYP liquid medium can be used to culture the strain. The lyophilized cell powder obtained by using the MYP liquid medium has good stability. The liquid culture with the MYP liquid medium can be used as a liquid starter for bread making. In this case, the stability of the bacterial liquid is improved by suspending the liquid in a 10 to 20% skim milk solution.

### EXAMPLES

The present invention is specifically described below referring to Examples, but is not limited only to these Examples.

### 1. Isolation of Lactobacillus Sanfranciscensis WB1006

The strain was isolated by gradually diluting a panettone starter used for bread making with sterilized saline, and applying and incubating the dilution in an isolation agar medium (e.g. MYP agar medium, 1% maltose-containing MRS agar medium) containing 10 ppm of cycloheximide and 10 ppm of sodium azide. The culture condition was at 25° C. and colonies were separated after 2 to 4-day culture.

### 2. Identification of Bacteriological Characteristics

The morphological characteristics of Lactobacillus sanfranciscensis WB1006 were identified in an MYP liquid medium. The MYP liquid medium was prepared by adding maltose (10 g), yeast extract (5 g), peptone (1 g), sodium acetate (1 g), sodium glutamate (1 g), magnesium sulfate (200 mg), manganese sulfate (20 mg), ferrous sulfate (10 mg), sodium chloride (10 mg), and Tween 80 (0.25 g) to water (1000 ml), and was adjusted to a pH of 6.6 with 1N NaOH.

After 24-hour culture in the MYP liquid medium, the bacterium was found to have a long rod shaped form with a size of 1.0-5.0×0.4 μm and was present singly or in chains. The bacterium was nonsporing, nonmotile, and gram positive. The bacteriological characteristics were identified according to “Experimental Manual of Lactic Acid Bacteria” (Asakura Publishing Co., Ltd.). Further, Bergey's Manual of Systematic Bacteriology Vol. 2 (1986) was used as the criteria for taxonomic identification. The identified bacteriological characteristics are listed below.

(1) Gram positive

(2) Rod shaped

(3) Nonmotile

(4) Nonsporing

(5) Facultative anaerobe

(6) Catalase negative

(7) Growth temperature range: 10° C. to 28° C. (optimum growth temperature: 25° C.)

(8) Growth pH range: 5.5 to 9.0

(9) Utilizing maltose to produce D(+)- and L(+)-lactic acids, ethanol and carbon dioxide.

In addition, the strain very slightly utilized glucose under some culture conditions.

### 3. Identification of Cultural Characteristics

Lactobacillus sanfranciscensis WB1006 of the present invention was assayed for cultural characteristics in the following media (1) to (3).

(1) 1% Maltose-containing MRS agar plate medium

The medium was prepared by adding 10 g of maltose and 15 g of agar to 1000 ml of Difco Lactobacilli MRS broth. After 3- or 4-day culture at 25° C., colonies were found to be circular, approximately 2 to 3 mm or less in diameter, convex in elevation, opaque grayish-white in color, and slightly dry.

(2) MYP liquid medium

After 24-hour culture at 25° C., cells grew to make the medium cloudy and to form a fluffy precipitate in the medium.

(3) MYP agar medium (stab culture)

The MYP agar medium was prepared by adding 15 g of agar to 1000 ml of the MYP liquid medium. Cells uniformly grew in a puncture in the medium.

### 4. Identification of Physiological and Biochemical Characteristics

The bacteriological characteristics were identified according to “Experimental Manual of Lactic Acid Bacteria” (Asakura Publishing Co., Ltd.). The identified physiological and biochemical characteristics are listed below.

The physiological and biochemical characteristics of the strain of the present invention are listed below.

(1) Growth temperature range: 10° C. to 28° C. (optimum growth temperature: 25° C.)

(2) Growth pH range: 5.5 to 9.0

(3) Relationship with oxygen:

Facultatively anaerobic. The strain can grow either in the presence of oxygen or under anaerobic conditions.

(4) Substances essential for growth:

Maltose, yeast extract and a fatty acid, in particular an unsaturated fatty acid are essentially required in the MYP liquid medium.

(5) Sugar fermentability:

The strain utilizes maltose to produce an acid and gas.

(6) Litmus milk: no change

(7) Reduction of nitrates: negative

(8) Gelatin is not liquefied.

(9) Urease: negative

(10) Catalase: negative

(11) Starch is not hydrolyzed.

(12) Products from maltose: L(+)- and D (+) -lactic acids, and ethanol

### 5. Gene Analysis

Lactobacillus sanfranciscensis WB1006 of the present invention was genetically analyzed in the following manner. The base sequence data of the 16S rRNA gene of Lactobacillus sanfranciscensis WB1006 was compared with the sequence data of a known species to identify the taxonomic position of Lactobacillus sanfranciscensis WB1006. The DNA was extracted according to a general method from a liquid culture after 24-hour culture in the MYP liquid medium at 25° C. The 16S rRNA gene analysis revealed that the 1564 by sequence of Lactobacillus sanfranciscensis WB1006 had 99.7% sequence identity to Lactobacillus sanfranciscensis JCM5668 (JAPAN COLLECTION OF MICROORGAMISMS, Independent Administrative Institution, RIKEN), which is the type strain of Lactobacillus sanfranciscensis.

6. Comparison between Lactobacillus Sanfranciscensis WB1006 and Type Strain

A comparison of the sugar utilization between the strain of the present invention and Lactobacillus sanfranciscensis JCM5668 showed that the strain of the present invention remarkably specifically utilized maltose as a carbon source and hardly utilized glucose under common culture conditions; and Lactobacillus sanfranciscensis JCM5668, on the other hand, utilized both maltose and glucose. In addition, the culture temperature range of the strain of the present invention was low and the growth temperature was 28° C. or lower. Particularly, the strain grew well at a temperature of 25° C. while the optimum growth temperature of Lactobacillus sanfranciscensis JCM5668 was 30° C. to 35° C. Based on the characteristics different from those of the known strain, the strain of the present invention was regarded as a novel strain and named Lactobacillus sanfranciscensis WB1006.

### Example 1

### (Bread Making)

The following materials were used in an MYP liquid medium.

Maltose: “maltose monohydrate” (Wako Pure Chemical Industries Ltd.)

Yeast Extract: “Yeast Extract” (Difco)

Peptone: “Peptone, Bacto TM” (Difco)

Sodium acetate: “sodium acetate trihydrate” (Wako Pure Chemical Industries Ltd.)

Sodium glutamate: “L-glutamic acid monosodium salt” (Wako Pure Chemical Industries Ltd.)

Magnesium sulfate: “magnesium sulfate heptahydrate” (Wako Pure Chemical Industries Ltd.)

Manganese sulfate: “Manganese (II) sulfate tetrahydrate” (Wako Pure Chemical Industries Ltd.)

Ferrous sulfate: “iron (II) sulfate heptahydrate” (Wako . Pure Chemical Industries Ltd.)

Sodium chloride: “sodium chloride” (Wako Pure Chemical Industries Ltd.)

Tween 80: “polyoxyethylene (20) sorbitan monooleate” (Wako Pure Chemical Industries Ltd.)

The following materials were used for bread making.

Flour: “Eagle” (Nippon Flour Mills Co., Ltd.)

Yeast: “US yeast” (Oriental Yeast Co., Ltd.)

Salt: “Salt” (The Salt Industry Center of Japan)

Sugar: “Granulated sugar GHC1” (Mitsui Sugar Co., Ltd.)

Skim milk powder: “Milfine” (JT Foods Co., Ltd.)

Shortening: “Premium short CF” (ADEKA Corp.)

Bread improver: “Dough natural GF” (Oriental Yeast Co., Ltd.)

A bread was made by the sponge and dough method. The materials in amounts shown in Table 1 were mixed, and the mixture was kneaded at 24° C. and allowed to ferment at 28° C. at 75 RH % for 12 hours. In this manner, a starter was obtained.

TABLE 1

| Materials | Part(s) by weight |
|---|---|
| Flour (bread flour) | 100 |
| Yeast | 0.15 |
| Liquid or powder of lactic acid bacterium | 1 |
| Water | 49 |

Next, a sponge was prepared by mixing the starter and the materials shown in Table 2. The sponge was kneaded at 24° C., while the mixing condition was controlled to a low speed for three minutes and a medium speed for one minute (L3M1). Subsequently, the sponge was allowed to ferment at 28° C. at 75RH % for four hours.

TABLE 2

| Materials | Part(s) by weight |
|---|---|
| Flour (bread flour) | 70 |
| Yeast | 2 |
| Bread improver | 0.1 |
| Starter prepared above | 20 |
| Water | 40 |

The sponge and the materials in amounts shown in Table 3 were mixed and the resulting dough was kneaded. After a 20-minute floor time, the dough was divided into 220g portions. These portions were shaped after a 20-minute bench time, and six of them were put into a double-loaf bread mold, allowed to ferment in a proofer (35° C., 75 RH %, 60 minutes) , and then baked (upper temperature: 200° C.; lower temperature: 230° C.; 32 minutes). In this manner, a bread was made. The dough was kneaded at 26° C., while the mixing condition was controlled to a low speed for three minutes, a medium speed for three minutes, and a high speed for one minute, and after the addition of the shortening, was controlled to a low speed for two minutes, a medium speed for two minutes, and a high speed for one minute.

TABLE 3

| Materials | Part(s) by weight |
|---|---|
| Sponge prepared above | 132.1 |
| Flour (bread flour) | 30 |
| Salt | 2 |
| Sugar | 5 |
| Skim milk powder | 2 |
| Shortening | 5 |
| Water | 28 |

### Comparative Example 1

### (Bread Making)

A bread was made in the same manner as in Example 1, except that the strain of the present invention in the starter of Example 1 was not added at all.

### Comparative Example 2

### (Bread Making)

A bread was made in the same manner as in Example 1, except that Lactobacillus sanfranciscensis JCM5668 (type strain) was used for the starter instead of the strain of the present invention used in Example 1.

### Test Example 1

### (Evaluation of Antifungal Performance of Lactic Acid Bacterium-containing Bread)

### [Test Method]

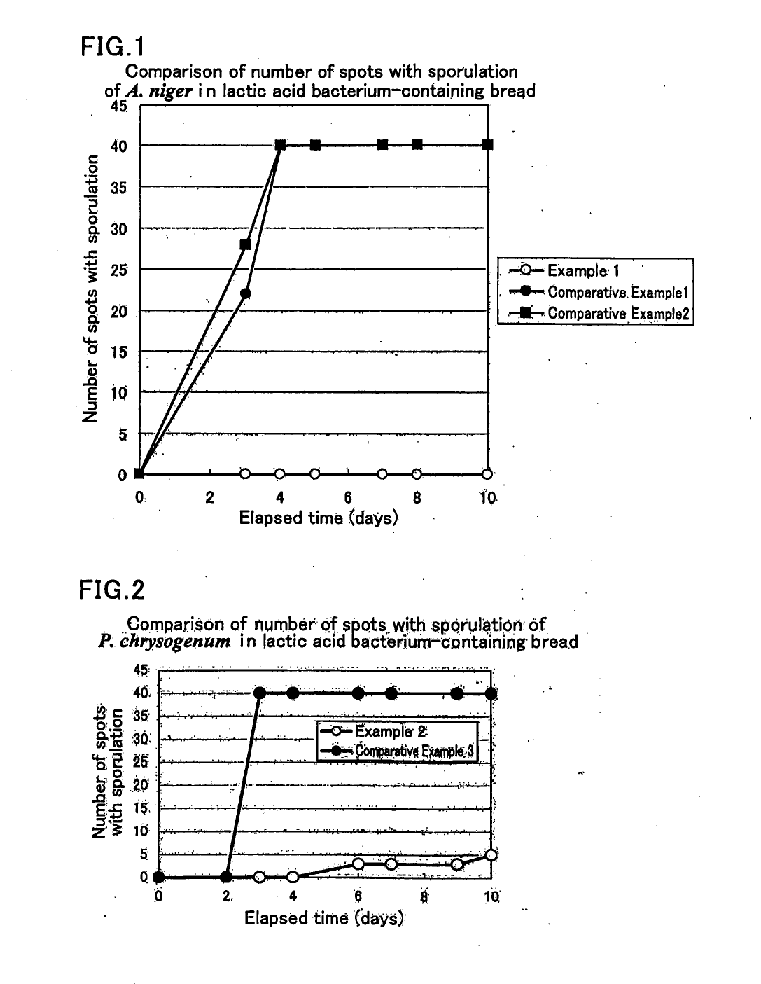

A compulsory fungal contamination test was performed on the breads made in Example 1 and Comparative Examples 1 and 2. The test fungal strain was a famous fungus, Aspergillus niger (hereinafter, abbreviated as A. niger). The breads were sliced, and about 50 fungal spores were inoculated at 40 spots. The number of spots where sporulation was observed was counted and the number of days until sporulation was recorded. Sporulation of the fungus was observed in the breads of Comparative Examples 1 and 2 after about three days from the contamination. In contrast, sporulation of the fungus was not observed in the bread of Example which contained the strain of the present invention, even after about 35 days from the contamination. The test results are shown in Table 4 and FIG. 1.

This test example demonstrated that the use of the strain of the present invention for a starter for bread provides an antifungal effect higher than that conventionally obtained.

TABLE 4

| Elapsed time | | Comparative | Comparative |
|---|---|---|---|
| (days) | Example 1 | Example 1 | Example 2 |
| 0 | 0 | 0 | 0 |
| 3 | 0 | 22 | 28 |
| 4 | 0 | 40 | 40 |
| 5 | 0 | 40 | 40 |
| 7 | 0 | 40 | 40 |
| 8 | 0 | 40 | 40 |
| 10 | 0 | 40 | 40 |

### Example 2

### (Bread Making)

The same MYP liquid medium and bread making materials as those of Example 1 were used in Example 2. A bread was made by the sponge and dough method. The materials in amounts shown in Table 5 were mixed, and the mixture was kneaded at 24° C. and allowed to ferment at 28° C. at 75 RH% for 12 hours. In this manner, a starter was obtained.

TABLE 5

| Materials | Part(s) by weight |
|---|---|
| Flour (bread flour) | 100 |
| Liquid or powder of lactic acid bacterium | 1 |
| Water | 120 |

Next, a sponge was prepared by mixing the starter of Table 5 and the other materials shown in Table 6. The sponge was kneaded at 24° C. while the mixing condition was controlled to a low speed for three minutes. Subsequently, the sponge was allowed to ferment at 28° C. at 75RH % for four hours.

TABLE 6

| Materials | Part(s) by weight |
|---|---|
| Flour (bread flour) | 70 |
| Yeast | 2 |
| Starter | 20 |
| Water | 40 |

The sponge of Table 6 and the materials in amounts shown in Table 7 were mixed and the resulting dough was kneaded. After the kneading, the dough was divided into 220-g portions. After a 20-minute bench time, these portions were shaped, filled into a mold, allowed to ferment in a proofer (35° C., 75 RH %, 60 minutes) , and then baked (lower temperature: 175° C.; upper temperature: 200° C.; 20 minutes). In this manner, a bread was made. The dough was kneaded at 26° C. while the mixing condition was controlled to a low speed for three minutes and a medium speed for two minutes, and after the addition of the shortening, was controlled to a low speed for two minutes and a medium speed for one minute.

TABLE 7

| Materials | Part(s) by weight |
|---|---|
| Sponge | 132 |
| Flour (bread flour) | 30 |
| Salt | 2 |
| Sugar | 5 |
| Bread improver | 0.2 |
| Skim milk powder | 2 |
| Shortening | 5 |
| Water | 28 |

### Comparative Example 3

### (Bread Making)

A bread was made in the same manner as in Examples 2, except that the strain of the present invention in the starter of Example 2 was not added at all.

### Test Example 2

### (Evaluation of Antifungal Performance of Lactic Acid Bacterium-containing Bread)

A compulsory fungal contamination test was performed on the breads made in Example 2 and Comparative Example 3. The test fungal strain was a famous fungus, Penicillium chrysogenum (hereinafter, abbreviated as P. chrysogenum). The antifungal performance was evaluated in the same manner as in Test Example 1. The first record of sporulation of the fungus was about three days after the contamination, and sporulation was observed at all the inoculated spots in the bread of Comparative Example 3. Regarding Example 2, the first record of sporulation of the fungus was about six days after the contamination, and sporulation was observed at a part of the spots even after seven to ten days. The increasing rate of contaminated spots of Example 2 was much slower than that of Comparative Example 3. The test results are shown in Table 8 and FIG. 2.

TABLE 8

| Elapsed time | | Comparative |
|---|---|---|
| (days) | Example 2 | Example 3 |
| 0 | 0 | 0 |
| 2 | 0 | 0 |
| 3 | 0 | 40 |
| 4 | 0 | 40 |
| 6 | 3 | 40 |
| 7 | 3 | 40 |
| 9 | 3 | 40 |
| 10 | 5 | 40 |

The growth inhibitory effect against Staphylococcus aureus, which is known as a food poisoning microorganism, was assayed using the bread made in Example 2, which contained the strain of the present invention, and the bread made in Comparative Example 3, which was free from the strain of the present invention.

### Test Example 3

(Growth Inhibitory Effect of Lactic Acid Bacterium-containing Bread Against Staphylococcus aureus)

### [Test Method]

The used test strain of Staphylococcus aureus was Staphylococcus aureus JCM2413 (hereinafter, abbreviated as Staphylococcus aureus).

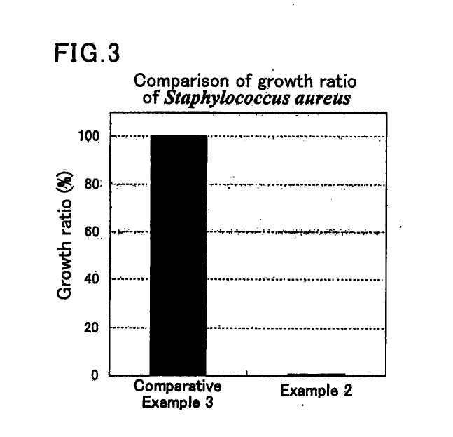

The breads of Comparative Example 3 and Example 2 were sliced, and the inner part of each slice was cut into square samples of 2.5 cm×2.5 cm (2.5 to 2.8 g/sample) . About 1000 colony forming units (CFU)/500 μL/sample of Staphylococcus aureus was inoculated. The breads with the test bacterium inoculated thereon were incubated in a sealed vessel at 35° C. for 24 hours. After the incubation, each sample was suspended in PBS (−) and viable cells were counted in a medium for isolating Staphylococci (Staphylococcus Medium No.110, Nissui Pharmaceutical Co. Ltd.). The growth in the lactic acid bacterium-free bread (Comparative Example 3) was taken as 100%, and the growth ratio of the bread containing the strain of the present invention (Example 2) was compared thereto. The results are shown in Table 9 and FIG. 3. The numbers of Staphylococcus aureus cells in the table are the averages of tested four samples (n=4).

TABLE 9

| | Number of Staphylococcus aureus cells | |
|---|---|---|
| | (CFU/sample) | |
| | Immediately after | |
| Examples | inoculation | After 24 hours |
| Comparative Example 3 | 1.02 × 10³ | 3.01 × 10⁸ |
| Example 2 | 1.02 × 10³ | 1.99 × 10⁶ * |

* Statistically significant difference (p < 0.05) from Comparative Example 3 (Student t-test)

Staphylococcus aureus grew to 3×10⁸ CFU/sample in Comparative Example 3. The number of Staphylococcus aureus cells in the bread containing the strain of the present invention of Example 2 was 2.0×10⁶ CFU/sample, and the growth ratio of Example 2 was less than 1/100 of the growth ratio of Comparative Example 3. This test revealed that the use of the strain of the present invention in bread results in inhibition of the growth of Staphylococcus aureus.

It is clear from Test Examples 1 to 3 that the strain of the present invention still has the growth inhibitory effect against fungi and Staphylococcus aureus even after a baking process for bread making in the case where the strain is used in a sponge.

### INDUSTRIAL APPLICABILITY

The strain of the present invention can effectively inhibit the growth of bacteria, such as Staphylococcus aureus, as well as fungi, is safe, does not influence the flavor and taste of foods, can be used in the form of a lyophilized powder that is excellent in storage stability, and enables anyone to easily make stable sourdough, bread and the like foods.

1. A strain of Lactobacillus sanfranciscensis FERM ABP-11246.

2. A strain, having the following bacteriological characteristics:
(1) gram positive;
(2) rod shaped;
(3) nonmotile;
(4) nonsporing;
(5) facultative anaerobe;
(6) catalase negative;
(7) a growth temperature range of from 10° C. to 28° C.;
(8) a growth pH range of from 5.5 to 9.0; and
(9) utilizing maltose to produce D(+)- and L(+)-lactic acids, ethanol, and carbon dioxide.

3. A culture of the strain of claim 1, a material comprising the strain, or a lyophilized powder of the strain.

4. The culture, the material, or the lyophilized powder of claim 3, wherein the strain is viable.

5. A method for culturing a strain, the method comprising: inoculating the strain of claim 1 in a medium, to obtain an inoculated strain; and
culturing the inoculated strain.

6. A food additive, comprising the strain of claim 1, a culture of the strain, a material comprising the strain, or a lyophilized powder of the strain.

7. The food additive of claim 6, which is still capable of inhibiting the growth of fungi and Staphylococcus aureus after heating treatment.

8. A food, comprising the food additive of claim 6.

9. The food of claim 8, obtained by fermenting a food material by the strain.

10. A method for producing a starter which produces a growth inhibitory effect against fungi and Staphylococcus aureus, the method comprising:
fermenting a mixture comprising a lactic acid bacterium, flour and water,
wherein the lactic acid bacterium mainly ferments a sugar other than glucose and slightly utilizes another sugar.

11. The method of claim 10, wherein the mixture further comprises a yeast.

12. The method of claim 11, wherein the lactic acid bacterium is capable of growing at a temperature of 10° C. to 15° C.

13. A method for producing a food, the method comprising:
producing a sponge with a starter produced by the method of claim 10; and
forming a final dough.

14. A culture of the strain of claim 2, a material comprising the strain, or a lyophilized powder of the strain.

15. The culture, the material, or the lyophilized powder of claim 14, wherein the strain is viable.

16. A method for culturing a strain, the method comprising:
inoculating the strain of claim 2 in a medium, to obtain an inoculated strain; and
culturing the inoculated strain.

17. A food additive, comprising the strain of claim 2, a culture of the strain, a material comprising the strain, or a lyophilized powder of the strain.

18. The food additive of claim 17, which is still capable of inhibiting the growth of fungi and Staphylococcus aureus after heating treatment.

19. A food, comprising the food additive of claim 7.

20. The food of claim 19, obtained by fermenting a food material by the strain.
